(19) United States
(12) Patent Application Publication
(10) Pub. No.: US 2018/0071231 A1
(43) Pub. Date: Mar. 15, 2018

(54) FORMULATION AND AEROSOL CANISTERS, INHALERS, AND THE LIKE CONTAINING THE FORMULATION

(71) Applicant: 3M INNOVATIVE PROPERTIES COMPANY, St. Paul, MN (US)

(72) Inventors: KEWU ZHU, SINGAPORE (SG); DAVID W. SCHULTZ, PINE SPRINGS, MN (US); JAMES B. DRAKE, HUDSON, WI (US); YU CHEN, SINGAPORE (SG); MEI-LING PAN, SINGAPORE (SG); ENG SENG YAP, SINGAPORE (SG)

(21) Appl. No.: 15/564,466

(22) PCT Filed: Apr. 7, 2016
(86) PCT No.: PCT/US16/26313

§ 371 Date: Oct. 5, 2017

Related U.S. Application Data
(60) Provisional application No. 62/145,838, filed on Apr. 10, 2015.

(51) Int. Cl. A61K 31/137; A61K 9/00; A61K 31/46; A61M 15/00

(52) U.S. Cl. CPC A61K 31/137; A61K 9/008; A61M 15/0065; A61M 15/009; A61K 31/46

(57) ABSTRACT

Compositions containing albuterol, ipratropium, and a propellant as well as aerosol canisters and inhalers, such as metered dose inhalers, containing such compositions.

### TECHNICAL FIELD

The present disclosure relates to formulations used for, as an example, an inhaled dosage form, as well as aerosol canisters, inhalers, metered dose inhalers, and the like containing the same.

### BACKGROUND

Albuterol compositions, particularly for inhalers are known in the art. Such compositions are not necessarily acceptable, particularly when a second active agent, such as ipratropium, is also included. In particular, prior art compositions are not always sufficiently stable for storage.

### SUMMARY

A composition can comprise particulate albuterol or a pharmaceutically acceptable salt or solvate thereof; particulate ipratropium or a pharmaceutically acceptable salt or solvate thereof; and at least one of 1,1,1,2,3,3,3-heptafluoropropane (also known as HFA-227) and 1,1,1,2-tetrafluoroethane (also known as HFA-134a).

### DETAILED DESCRIPTION

Throughout this disclosure, singular forms such as “a,” “an,” and “the” are often used for convenience; however, it should be understood that the singular forms are meant to include the plural unless the singular alone is explicitly specified or is clearly indicated by the context.

Some terms used in this application have special meanings, as defined herein. All other terms will be known to the skilled artisan, and are to be afforded the meaning that a person of skill in the art at the time of the invention would have given them.

Elements in this specification that are referred to as “common,” “commonly used,” and the like, should be understood to be common within the context of the compositions, articles, such as inhalers and metered dose inhalers, and methods of this disclosure; this terminology is not used to mean that these features are present, much less common, in the prior art. Unless otherwise specified, only the Background section of this Application refers to the prior art.

The “particle size” of a single particle is the size of the smallest hypothetical hollow sphere that could encapsulate the particle.

The “mass median diameter” of a plurality of particles refers to the value for a particle diameter at which 50% of the mass of particles in the plurality of particles have a particle size smaller than the value and 50% of the mass of particles in the plurality of particle have a particle size greater than the value.

The “canister size” of a plurality of particles refers to the mass mean diameter of the plurality of particles when the formulation is prepared.

The “ex-actuator size” of a plurality of particles refers to the aerodynamic mass median diameter of the plurality of particles after the plurality of particles has passed through the actuator of an inhaler, such as a metered dose inhaler, as measured by the procedure described in the United States Pharmacopeia <601>.

When the concentration of albuterol is discussed in this application, for convenience it is referred to in terms of the concentration of the form of albuterol that is most commonly used in this disclosure, that is, albuterol sulfate. It should therefore be understood that if another form or salt of albuterol is used, the concentration of that other form or salt should be calculated on a basis relative to albuterol sulfate. A person of ordinary skill in the relevant arts can easily perform this calculation by comparing the molecular weight of the form or salt of albuterol that is used to the molecular weight of albuterol sulfate.

When the concentration of ipratropium is discussed in this application, for convenience it is referred to in terms of the concentration of the form of ipratropium that is most commonly used in this disclosure, ipratropium bromide monohydrate. It should therefore be understood that if another form, hydrate, or salt of ipratropium is used, the concentration of that other form or salt should be calculated on a basis relative to ipratropium bromide monohydrate. A person of ordinary skill in the relevant arts can easily perform this calculation by comparing the molecular weight of the form, hydrate, or salt of ipratropium that is used to the molecular weight of ipratropium bromide monohydrate

Formulation

A pharmaceutical formulation can comprise particulate albuterol. Albuterol is sometimes known as salbutamol. The albuterol can be a free base, but is more typically in the form of one or more physiologically acceptable salts or solvates. Albuterol sulfate is most common.

The albuterol, such as albuterol sulfate, is in particulate form. The canister size of the particles of albuterol, such as albuterol sulfate, can be any suitable canister size. Exemplary suitable canister sizes can be no less than 1 micrometer no less than 1.5 micrometers, no less than 2 micrometers, no less than 2.5 micrometers, no less than 3 micrometers, no less than 3.5 micrometers, no less than 4 micrometers, or no less than 4.5 micrometers. Exemplary suitable canister sizes can also be no greater than 5 micrometers, no greater than 4.5 micrometers, no greater than 4.0 micrometers, no greater than 3.5 micrometers, no greater than 3.0 micrometers, no greater than 2.5 micrometers, no greater than 2.0 micrometers, or no greater than 1.5 micrometers. 1 micrometer to 5 micrometers is common.

The ex-actuator size of the albuterol particles, such as albuterol sulfate particles, can be any suitable ex-actuator size. Exemplary suitable ex-actuator sizes can be no less than 1 micrometer no less than 1.5 micrometers, no less than 2 micrometers, no less than 2.5 micrometers, no less than 3 micrometers, no less than 3.5 micrometers, no less than 4 micrometers, or no less than 4.5 micrometers. Exemplary suitable ex-actuator sizes can also be no greater than 5 micrometers, no greater than 4.5 micrometers, no greater than 4.0 micrometers, no greater than 3.5 micrometers, no greater than 3.0 micrometers, no greater than 2.5 micrometers, no greater than 2.0 micrometers, or no greater than 1.5 micrometers. 1 micrometer to 5 micrometers is common.

The albuterol, such as albuterol sulfate, can be present in any suitable concentration in the formulation. When the concentration of albuterol is expressed in terms of mg/mL, then the concentration of albuterol can be no less than 1.5, no less than 1.6, no less than 1.7, no less than 1.8, no less than 1.9, no less than 2.0, no less than 2.1, no less than 2.2, no less than 2.3, no less than 2.4, no less than 2.5, no less than 2.6, no less than 2.7, no less than 2.8, no less than 2.9, no less than 3.0, no less than 3.1, no less than 3.2, no less than 3.3, no less than 3.4, no less than 3.5, no less than 3.6, no less than 3.7, no less than 3.8, no less than 3.9, no less than 4, no less than 4.1, no less than 4.2, no less than 4.3, no less than 4.4, no less than 4.5, no less than 4.6, no less than 4.8, no less than 4.9, no less than 5.0, no less than 5.1, no less than 5.1, no less than 5.2, no less than 5.3, no less than 5.4, no less than 5.5, no less than 5.6, no less than 5.7, no less than 5.8, no less than 5.9, no less than 6.0, no less than 6.1, no less than 6.2, no less than 6.3, no less than 6.4, no less than 6.5, no less than 6.6, no less than 6.7, no less than 6.8, no less than 6.9, no less than 7.0, no less than 7.1, no less than 7.2, no less than 7.3, no less than 7.4, no less than 7.5, no less than 7.6, no less than 7.7, no less than 7.8, no less than 7.9, no less than 8.0, no less than 8.1, no less than 8.2, no less than 8.3, no less than 8.4, no less than 8.5, no less than 8.6, no less than 8.7, no less than 8.8, no less than 8.9, no less than 9.0, no less than 9.1, no less than 9.2, no less than 9.3, no less than 9.4, no less than 9.5, no less than 9.6, no less than 9.7, no less than 9.8, no less than 9.9, no less than 10.0, no less than 10.1, no less than 10.2, no less than 10.3, no less than 10.4, no less than 10.5, no less than 10.6, no less than 10.7, no less than 10.8, no less than 10.9, or no less than 11. Also on a mg/mL basis, the concentration of albuterol can be no greater than 11, no greater than 10.9, no greater than 10.8, no greater than 10.7, no greater than 10.6, no greater than 10.5, no greater than 10.4, no greater than 10.3, no greater than 10.2, no greater than 10.1, no greater than 10.0, no greater than 9.9, no greater than 9.8, no greater than 9.7, no greater than 9.6, no greater than 9.5, no greater than 9.4, no greater than 9.3, no greater than 9.2, no greater than 9.1, no greater than 9.0, no greater than 8.9, no greater than 8.8, no greater than 8.7, no greater than 8.6, no greater than 8.5, no greater than 8.4, no greater than 8.3, no greater than 8.2, no greater than 8.1, no greater than 8.0, no greater than 7.9, no greater than 7.8, no greater than 7.7, no greater than 7.6, no greater than 7.5, no greater than 7.4, no greater than 7.3, no greater than 7.2, no greater than 7.1, no greater than 7.0, no greater than 6.9, no greater than 6.8, no greater than 6.7, no greater than 6.6, no greater than 6.5, no greater than 6.4, no greater than 6.3, no greater than 6.2, no greater than 6.1, no greater than 6.0, no greater than 5.9, no greater than 5.8, no greater than 5.7, no greater than 5.6, no greater than 5.5, no greater than 5.4, no greater than 5.3, no greater than 5.2, no greater than 5.1, no greater than 5.0, no greater than 4.9, no greater than 4.8, no greater than 4.7, no greater than 4.6, no greater than 4.5, no greater than 4.4, no greater than 4.3, no greater than 4.2, or no greater than 4.1. One typical range is from 4 mg/mL to 11 mg/mL. Another typical range is from 4.19 mg/mL to 10.56 mg/mL. For some applications, a concentration of 4.13 mg/mL is employed. For other applications, a concentration of 5.28 mg/mL is employed. For still other applications, a concentration of 10.56 mg/mL is employed.

Ipratopium, particularly ipratropium bromide and more particularly ipratropium bromide monohydrate can also be included. The ipratropium, such as ipratopium bromide or ipratropium bromide monohydrate, is also in particulate form. The canister size of the particles of ipratropium, such as ipratopium bromide or ipratropium bromide monohydrate, can be any suitable canister size. Exemplary suitable canister sizes can be no less than 1 micrometer no less than 1.5 micrometers, no less than 2 micrometers, no less than 2.5 micrometers, no less than 3 micrometers, no less than 3.5 micrometers, no less than 4 micrometers, or no less than 4.5 micrometers. Exemplary suitable canister sizes can also be no greater than 5 micrometers, no greater than 4.5 micrometers, no greater than 4.0 micrometers, no greater than 3.5 micrometers, no greater than 3.0 micrometers, no greater than 2.5 micrometers, no greater than 2.0 micrometers, or no greater than 1.5 micrometers. 1 micrometer to 5 micrometers is common.

The ex-actuator size of the ipratropium particles, such as ipratopium bromide or ipratropium bromide monohydrate, can be any suitable ex-actuator size. Exemplary suitable ex-actuator sizes can be no less than 1 micrometer no less than 1.5 micrometers, no less than 2 micrometers, no less than 2.5 micrometers, no less than 3 micrometers, no less than 3.5 micrometers, no less than 4 micrometers, or no less than 4.5 micrometers. Exemplary suitable ex-actuator sizes can also be no greater than 5 micrometers, no greater than 4.5 micrometers, no greater than 4.0 micrometers, no greater than 3.5 micrometers, no greater than 3.0 micrometers, no greater than 2.5 micrometers, no greater than 2.0 micrometers, or no greater than 1.5 micrometers. 1 micrometer to 5 micrometers is common.

The ipratropium can be used in any suitable concentration. On a mg/mL basis, typical concentrations are no less than 0.3, no less than 0.4, no less than 0.5, no less than 0.6, no less than 0.7, no less than 0.8, no less than 0.9, no less than 1.0, no less than 1.1, no less than 1.2, no less than 1.3, no less than 1.4, no less than 1.5, no less than 1.6, no less than 1.7, no less than 1.8, no less than 1.9, or no less than 2.0. Typical concentrations are also no greater than 2.0, no greater than 1.9, no greater than 1.8, no greater than 1.7, no greater than 1.6, no greater than 1.5, no greater than 1.4, no greater than 1.3, no greater than 1.2, no greater than 1.1, no greater than 1.0, no greater than 0.9, no greater than 0.8, no greater than 0.7, no greater than 0.6, or no greater than 0.5. Common concentrations are from 0.5 mg/mL to 2 mg/mL, such as from 0.69 mg/mL to 1.76 mg/mL. For some applications, a concentration of 0.69 mg/mL is used. For other applications, a concentration of 0.88 mg/mL is used. For still other applications, a concentration of 1.76 mg/mL is used.

A propellant can also be included in the formulation. The propellant is typically 1,1,1,2,3,3,3,-heptafluoropropane, 1,1,1,2-tetrafluoroethane, or a combination thereof. The propellant typically also serves to as a dispersant for the particles of albuterol, such as albuterol sulfate, and ipratropium, such as ipratropium bromide or ipratropium bromide monohydrate.

The particles of albuterol, such as albuterol sulfate, and ipratropium, such as ipratropium bromide or ipratropium bromide monohydrate, are typically not dissolved in the formulation. Instead, the particles of albuterol, such as albuterol sulfate, and ipratropium, such as ipratropium bromide or ipratropium bromide monohydrate are suspended in the propellant.

In order to facilitate this suspension, additional components can be added to the formulation. One such additional component is ethanol. Another such additional component is a surfactant. These additional components are not required unless otherwise specified.

When ethanol is used, it is typically employed in relatively low concentrations. On a weight percent basis, the amount of ethanol used, if any, is typically no greater than 5, no greater than 4.9, no greater than 4.8, no greater than 4.7, no greater than 4.6, no greater than 4.5, no greater than 4.4, no greater than 4.3, no greater than 4.2, no greater than 4.1, no greater than 4.0, no greater than 3.9, no greater than 3.8, no greater than 3.7, no greater than 3.6, no greater than 3.5, no greater than 3.4, no greater than 3.3, no greater than 3.2, no greater than 3.1, no greater than 3.0, no greater than 2.9, no greater than 2.8, no greater than 2.7, no greater than 2.6, no greater than 2.5, no greater than 2.4, no greater than 2.3, no greater than 2.2, no greater than 2.1, no greater than 2.0, no greater than 1.9, no greater than 1.8, no greater than 1.7, no greater than 1.6, no greater than 1.5, no greater than 1.4, no greater than 1.3, no greater than 1.2, no greater than 1.1, no greater than 1.0, no greater than 0.9, no greater than 0.8, no greater than 0.7, no greater than 0.6, or no greater than 0.5. On a weight percent basis, the amount of ethanol used, if any, is typically no less than 0.5, no less than 0.6, no less than 0.7, no less than 0.8, no less than 0.9, no less than 1.0, no less than 1.1, no less than 1.1, no less than 1.2, no less than 1.3, no less than 1.4, no less than 1.5, no less than 1.6, no less than 1.7, no less than 1.8, no less than 1.9, no less than 2.0, no less than 2.1, no less than 2.2, no less than 2.3, no less than 2.4, no less than 2.5, no less than 2.6, no less than 2.7, no less than 2.8, no less than 2.9, no less than 3.0, no less than 3.1, no less than 3.2, no less than 3.3, no less than 3 .4, no less than 3.5, no less than 3.6, no less than 3.7, no less than 3.8, no less than 3.9, no less than 4 .0, no less than 4.1, no less than 4.2, no less than 4.3, no less than 4.4, no less than 4.5, no less than 4.6, no less than 4.7, no less than 4.8, no less than 4.9, or no less than 5.0. Typical ranges of ethanol concentration, in those cases when ethanol is included, are from 0.1 wt. % to 5 wt. %, such as from 0.5 wt. % to 4 wt. %. In some cases, an ethanol concentration of 1 wt. % is employed.

One or more surfactant can also be used to facilitate suspension of the particles in the formulation. However, surfactant-free formulations can be advantageous for some purposes, and surfactant is not required unless otherwise specified.

Any pharmaceutically acceptable surfactant can be used. Most such surfactants are suitable for use with an inhaler. Typical surfactants include oleic acid, sorbitan monooleate, sorbitan trioleate, soya lecithin, polyethylene glycol, polyvinylpyrrolidone, or combinations thereof. Oleic, polyvinylpyrrolidone, or a combination thereof is most common. A combination of polyvinylpyrrolidone and polyethylene glycol is also commonly employed. When polyvinylpyrrolidone is employed, it can have any suitable molecular weight. Examples of suitable weight average molecular weights are from 10 to 100 kilodaltons, typically from 10 to 50, 10 to 40, 10 to 30 or 10 to 20 kilodaltons. When polyethylene glycol is employed, it can be any suitable grade. PEG 100 and PEG 300 are most commonly employed.

When used, the surfactant is typically present, on a weight percent basis, in an amount no less than 0.0001, no less than 0.01, no less than 0.02, no less than 0.03, no less than 0.04, no less than 0.05, no less than 0.06, no less than 0.07, no less than 0.08, no less than 0.09, no less than 0.10, no less than 0.11, no less than 0.12, no less than 0.13, no less than 0.14, no less than 0.15, no less than 0.16, no less than 0.17, no less than 0.18, no less than 0.19, no less than 0.2, no less than 0.21, no less than 0.22, no less than 0.23, no less than 0.24, no less than 0.25, no less than 0.26, no less than 0.27, no less than 0.28, no less than 0.29, no less than 0.3, no less than 0.4, no less than 0.5, no less than 0.6, no less than 0.7, no less than 0.8, no less than 0.9, or no less than 1. The surfactant is also typically present, on a weight percent basis, in an amount no greater than 1, no greater than 0.9, no greater than 0.8, no greater than 0.7, no greater than 0.6, no greater than 0.5, no greater than 0.4, no greater than 0.3, no greater than 0.29, no greater than 0.28, no greater than 0.27, no greater than 0.26, no greater than 0.25, no greater than 0.24, no greater than 0.23, no greater than 0.22, no greater than 0.21, no greater than 0.20, no greater than 0.19, no greater than 0.18, no greater than 0.17, no greater than 0.16, no greater than 0.15, no greater than 0.14, no greater than 0.13, no greater than 0.12, no greater than 0.11, no greater than 0.10, no greater than 0.09, no greater than 0.08, no greater than 0.07, no greater than 0.06, no greater than 0.05, no greater than 0.04, no greater than 0.03, no greater than 0.02, or no greater than 0.01. Concentration ranges can be from 0.0001 wt. % to 1 wt. %, such as 0.001 wt. % to 0.1 wt. %. Particular applications use 0.01 wt. % surfactant.

Particularly, oleic acid can be used in any of the abovementioned concentrations. Particularly, polyvinylpyrrolidone can be used in any of the abovementioned concentrations. Particularly, a combination of polyethylene glycol and polyvinylpyrrolidone can be used in any of the abovementioned concentrations. Particularly, sorbitan trioleate can be used in any of the abovementioned concentrations.

The formulations as described herein can be particularly advantageous because they can stabilize the albuterol and ipratropium contained therein. Stability of formulations of this type can be measured by comparing the ex-actuator particle size of albuterol, ipratropium, or both, immediately after filling the canister to the ex-actuator particle size of the same medicament after storage under specified conditions for a specified time. Under this comparison, a smaller change in ex-actuator particle size relates to a higher stability, whereas a larger change in ex-actuator particle size relates to a lower stability.

One particular set of conditions under which stability can be measured is storage of the pharmaceutical formulation in a canister is a particular temperature and a particular relative humidity, such as a temperature of 40° C. and a relative humidity of 75%. Stability can be measured after a particular storage time. A typical storage time is 6 months. A formulation, such as any formulation described herein, can be considered to have good stability if there is a sufficiently small change in fine particle mass at such particular temperatures and particular relative humidity. Fine particle mass can be determined using a Next Generation Impactor (NG) instrument, procedure, and calculation, examples of which are described in detail in the Examples section of this disclosure. A sufficiently small change in fine particle mass can be, for example, a change that is no greater than 15%, no greater than 14%, no greater than 13%, no greater than 12%, no greater than 11%, no greater than 10%, no greater than 9%, no greater than 8%, no greater than 7%, no greater than 6%, no greater than 5%, no greater than 4%, no greater than 3%, no greater than 2%, or no greater than 1%. Typically, a change of no greater than 5% is adequate, although greater change may be acceptable for some applications and less change may be required for others.

Alternatively, a formulation, such as any formulation described herein, can be considered to have good stability if there is a sufficiently small change in ex-actuator particle size at such particular temperatures and particular relative humidity. A sufficiently small change in ex-actuator particle size can be, for example, a change that is no greater than 15%, no greater than 14%, no greater than 13%, no greater than 12%, no greater than 11%, no greater than 10%, no greater than 9%, no greater than 8%, no greater than 7%, no greater than 6%, no greater than 5%, no greater than 4%, no greater than 3%, no greater than 2%, or no greater than 1%. Typically, a change of no greater than 5% is adequate, although greater change may be acceptable for some applications and less change may be required for others.

Any of the above-described formulations can be used with any type of inhaler. Metered dose inhalers are most common. When the inhaler is a metered dose inhaler, any metered dose inhaler can be employed. Suitable metered dose inhalers are known in the art.

Typical metered dose inhalers for the pharmaceutical formulations described herein contain an aerosol canister fitted with a valve. The canister can have any suitable volume. The brimful capacity canister will depend on the volume of the formulation that is used to fill the canister. In typical applications, the canister will have a volume from 5 mL to 500 mL, such as, for example 10 mL to 500 mL, 25 mL to 400 mL, 5 mL to 50 mL, 8 mL to 30 mL, 10 mL to 25 mL, or 50 to 250 mL. The canister will often have sufficient volume to contain enough medicament for delivering an appropriate number of doses. The appropriate number of doses is discussed herein. The valve is typically affixed, or crimpled, onto the canister by way of a cap or ferrule. The cap or ferrule is often made of aluminum or an aluminum alloy, which is typically part of the valve assembly. One or more seals can be located between the canister and the ferrule. The seals can be one or more of O-ring seals, gasket seals, and the like. The valve is typically a metered dose valve. Typical valve sizes range from 20 microliters to 35 microliters. Specific valve size that are commonly employed include 25, 50, 60, and 63 microliter valve sizes.

The container and valve typically include an actuator. Most actuators have a patient port, which is typically a mouthpiece, for delivering the formulation contained in the canister. The patient port can be configured in a variety of ways depending on the intended destination of the formulation. For example, a patient port designed for administration to the nasal cavities will generally have an upward slope to direct the formulation to the nose. The actuator is most commonly made out of a plastic material. Typical plastic materials for this purpose include at least one of polyethylene and polypropylene. Typical MDIs have an actuator with an orifice diameter. Any suitable orifice diameter can be used. Typical orifice diameters are from 0.2 mm to 0.65 mm. Typical orifice jet length is from 0.5 mm to 1 mm. Specific examples include orifice diameters of 0.4 mm, 0.5 mm, or 0.6 mm, any of which can have an orifice jet length of 0.8 mm.

A metered dose valve is typically present, and is often located at least partially within the canister and at least partially in communication with the actuator. Typical metered dose valves include a metering chamber that is at least partially defined by an inner valve body through which a valve stem passes. The valve stem can be biased outwardly by a compression spring to be in a sliding sealing engagement with an inner tank seal and outer diaphragm seal. The valve can also include a second valve body in the form of a body emptier. The inner valve body, which is sometimes referred to as the primary valve body, defines, in part, the metering chamber. The second valve body, which is sometimes referred to as the secondary valve body, defines, in part, a pre-metering region (sometimes called a pre-metering chamber) in addition to serving as a bottle emptier. The outer walls of the portion of the metered dose valve that are located within the canister, as well as the inner walls of the canister, defined a formulation chamber for containing the pharmaceutical formulation.

In use, the pharmaceutical formulation passes from the formulation chamber into the metering chamber. In moving to the metering chamber, the formulation can pass into the above-mentioned pre-metering chamber through an annular space between the secondary valve body (or a flange of the secondary valve body) and the primary valve body. Pressing the valve stem towards the interior of the container actuates the valve, which allows the pharmaceutical formulation to pass from the pre-metering chamber through a side hole in the valve stem, through an outlet in the valve stem, to an actuator nozzle, and finally through the patient port to the patient. When the valve stem is released, the pharmaceutical formulation enters the valve, typically to the pre-metering chamber, through an annular space and then travels to the metering chamber.

The pharmaceutical formulation can be placed into the canister by any known method. The two most common methods are cold filling and pressure filling. In a cold filling process, the pharmaceutical formulation is chilled to an appropriate temperature, which is typically −50° C. to −60° C. for formulations that use propellant HFA 134a, HFA 227, or a combination thereof, and added to the canister. The metered dose valve is subsequently crimped onto the canister. When the canister warms to ambient temperature, the vapor pressure associated with the pharmaceutical formulation increases thereby providing an appropriate pressure within the canister.

In a pressure filling method, the metered dose valve can be first crimped onto the empty canister. Subsequently, the formulation can be added through the valve into the container by way of applied pressure. Alternatively, all of the non-volatile components can be first added to the empty canister before crimping the valve onto the canister. The propellant can then be added through the valve into the canister by way of applied pressure.

Upon actuation, typical inhalers, such as metered dose inhalers, that are filled with any one of the formulations described herein can produce a fine particle mass of ipratropium, particularly ipratropium bromide or ipratropium bromide monohydrate that is from 3 mcg to 20 mcg per actuation and a fine particle mass of albuterol, particularly albuterol sulfate, that is from 16 mcg to 1116 mcg per actuation. In particular cases, inhalers, such as metered dose inhalers, produce a fine particle mass of ipratropium, particularly ipratropium bromide or ipratropium bromide monohydrate that is from 5 mcg to 15 mcg, and a fine particle mass of albuterol, particularly albuterol sulfate, that is from 55 mcg to 75 mcg per actuation. Fine particle mass can be calculated by the procedure described in the Experimental section of this disclosure.

The fine particle masses discussed above will typically correspond to a fine particle fraction of ipratropium, particularly ipratropium bromide or ipratropium bromide monohydrate and of albuterol, particularly albuterol sulfate, that is from 20% to 65%, which can be from 20% to 40% in particular cases, or from 25% to 35% in more particular cases. Fine particle fraction can be calculated by the procedure described in the experimental section of this disclosure.

Typical inhalers, such as metered dose inhalers, are designed to deliver a specified number of doses of the pharmaceutical formulation. In most cases, the specified number of doses is from 30 to 400, such as from 120 to 250. One commonly employed metered dose inhaler is designed to provide 120 doses; this can be employed with any of the formulations or inhaler types described herein. Another commonly employed metered dose inhaler is designed to provide 240 doses; this can be employed with any of the formulations or inhaler types described herein.

The inhaler, particularly when it is a metered dose inhaler, can contain a dose counter for counting the number of doses. Suitable dose counters are known in the art, and are described in, for example, U.S. Pat. No. 8,740,014, U.S. Pat. No. 8,479,732, US20120234317, and U.S. Pat. No. 8,814,035, all of which are incorporated by reference for their disclosures of dose counters.

One exemplary dose counter, which is described in detail in U.S. Pat. No. 8,740,014 (which is hereby incorporated by reference for its disclosure of the dose counter) has a fixed ratchet element and a trigger element that is constructed and arranged to undergo reciprocal movement coordinated with the reciprocal movement between an actuation element in an inhaler and the dose counter. The reciprocal movement typically comprises an outward stroke (outward being with respect to the inhaler) and a return stroke. The return stroke returns the trigger element to the position that it was in prior to the outward stroke. A counter element is also included in this type of dose counter. The counter element is constructed and arranged to undergo a predetermined counting movement each time a dose is dispensed. The counter element is biased towards the fixed ratchet and trigger elements and is capable of counting motion in a direction that is substantially orthogonal to the direction of the reciprocal movement of the trigger element.

The counter element in the above-described dose counter comprises a first region for interacting with the trigger member. The first region comprises at least one inclined surface that is engaged by the trigger member during the outward stroke of the trigger member. This engagement during the outward stroke causes the counter element to undergo a counting motion. The counter element also comprises a second region for interacting with the ratchet member. The second region comprises at least one inclined surface that is engaged by the ratchet element during the return stroke of the trigger element causing the counter element to undergo a further counting motion, thereby completing a counting movement. The counter element is normally in the form of a counter ring, and is advanced partially on the outward stroke of the trigger element, and partially on the return stroke of the trigger element. As the outward stroke of the trigger typically corresponds to the depression of a valve stem that causes firing of the valve (and, in the case of a metered dose inhaler, also meters the contents) and the return stroke typically corresponds to the return of the valve stem to its resting position, this dose counter allows for precise counting of doses.

Another suitable dose counter, which is described in detail in U.S. Pat. No. 8,479,732 (which is incorporated by reference for its disclosure of dose counters) is specially adapted for use with a metered dose inhaler. This dose counter includes a first count indicator having a first indicia bearing surface. The first count indicator is rotatable about a first axis. The dose counter also includes a second count indicator having a second indicia bearing surface. The second count indicator is rotatable about a second axis. The first and second axes are disposed such that they form an obtuse angle. The obtuse angle mentioned above can be any obtuse angle, but is advantageously 125 to 145 degrees. The obtuse angle permits the first and second indicia bearing surface to align at a common viewing area to collectively present at least a portion of a medication dosage count. One or both of the first and second indicia bearing surfaces can be marked with digits, such that when viewed together through the viewing area the numbers provide a dose count. For example, one of the first and second indicia bearing surface may have “hundreds” and “tens” place digits, and the other with “ones” place digits, such that when read together the two indicia bearing surfaces provide a number between 000 and 999 that represents the dose count.

Yet another suitable dose counter is described in US 20120234317 (hereby incorporated by reference for its disclosure of dose counters). Such a dose counter includes a counter element that undergoes a predetermined counting motion each time a dose is dispensed. The counting motion is typically vertical or essentially vertical. A count indicating element is also included. The count indicating element, which undergoes a predetermined count indicating motion each time a dose is dispensed, includes a first region that interacts with the counter element.

The counter element has regions for interacting with the count indicating element. Specifically, the counter element comprises a first region that interacts with a count indicating element. The first region includes at least one surface that it engaged with at least one surface of the first region of the aforementioned count indicating element. The first region of the counter element and the first surface of the count inducing element are disposed such that the count indicating member completes a count indicating motion in coordination with the counting motion of the counter element, during and induced by the movement of the counter element, the count inducing element undergoes a rotational or essentially rotational movement. In practice, the first region of the counter element or the counter indicating element can comprise, for example, one or more channels. A first region of the other element can comprise one or more protrusions adapted to engage with said one or more channels.

Yet another dose counter is described in U.S. Pat. No. 8,814,035 (hereby incorporated by reference for its disclosure of dose counters). Such a dose counter is specially adapted for use with an inhaler with a reciprocal actuator operating along a first axis. The dose counter includes an indicator element that is rotatable about a second axis. The indicator element is adapted to undergo one or more predetermined count-indicating motions when one or more doses are dispensed. The second axis is at an obtuse angle with respect to the first axis. The dose counter also contains a worm rotatable about a worm axis. The worm is adapted to drive the indicator element. It may do this, for example, by containing a region that interacts with and enmeshes with a region of the indicator element. The worm axis and the second axis do not intersect and are not aligned in a perpendicular manner. The worm axis is also, in most cases, not disposed in coaxial alignment with the first axis. However, the first and second axes may intersect.

At least one of the various internal components of an inhaler, such as a metered dose inhaler, as described herein, such as one or more of the canister, valve, gaskets, seals, O-rings, and the like, can be coated with one or more coatings. Some of these coatings provide a low surface energy. Such coatings are not required because they are not necessary for the successful operation of all inhalers.

Some coatings that can be used are described in U.S. Pat. No. 8,414,956, U.S. Pat. No. 8,815,325 and United States Patent Application Number US20120097159, all of which are incorporated by reference for their disclosure of coatings for inhalers and inhaler components.

A first acceptable coating can be provided by the following method:
a) providing one or more component of the inhaler, such as the metered dose inhaler,
b) providing a primer composition comprising a silane having two or more reactive silane groups separated by an organic linker group,
c) providing a coating composition comprising an at least partially fluorinated compound,
d) applying the primer composition to at least a portion of the surface of the component,
e) applying the coating composition to the portion of the surface of the component after application of the primer composition.

The at least partially fluorinated compound will usually comprise one or more reactive functional groups, with the or each one reactive functional group usually being a reactive silane group, for example a hydrolysable silane group or a hydroxysilane group. Such reactive silane groups allow reaction of the partially fluorinated compound with one or more of the reactive silane groups of the primer. Often such reaction will be a condensation reaction.

One exemplary silane that can be used has the formula

X₃₋ₘ(R¹)ₘSi—Q—Si(R²)ₖX₃₋ₖ

wherein R¹ and R² are independently selected univalent groups, X is a hydrolysable or hydroxy group, m and k are independently 0, 1, or 2 and Q is a divalent organic linking group.

Useful examples of such silanes include one or a mixture of two or more of 1,2-bis(trialkoxysilyl)ethane, 1,6-bis(trialkoxysilyl)hexane, 1,8-bis(trialkoxysilyl)octane, 1,4-bis(trialkoxysilylethyl)benzene, bis(trialkoxysilyl)itaconate, and 4,4′-bis(trialkoxysilyl)-1,1′-diphenyl, wherein any trialkoxy group may be independently trimethoxy or triethoxy.

The coating solvent usually comprises an alcohol or a hydrofluoroether.

If the coating solvent is an alcohol, preferred alcohols are C₁ to C₄ alcohols, in particular, an alcohol selected from ethanol, n-propanol, or iso-propanol or a mixture of two or more of these alcohols.

If the coating solvent is an hydrofluoroether, it is preferred if the coating solvent comprises a C₄ to C₁₀ hydrofluoroether. Generally, the hydrofluoroether will be of formula

C_{g}F_{2g+1}OCₕH₂ₕ₊₁

wherein g is 2, 3, 4, 5, or 6 and h is 1, 2, 3 or 4. Examples of suitable hydrofluoroethers include those selected from the group consisting of methyl heptafluoropropylether, ethyl heptafluoropropylether, methyl nonafluorobutylether, ethyl nonafluorobutylether and mixtures thereof.

The polyfluoropolyether silane is typically of the formula

R^{f}Q¹ᵥ[Q²_{w}—[C(R⁴)₂—Si(X)₃₋ₓ(R⁵)ₓ]_{y}]_{z}

wherein:

R^{f} is a polyfluoropolyether moiety;
Q¹ is a trivalent linking group;
each Q² is an independently selected organic divalent or trivalent linking group;

each R⁴ is independently hydrogen or a C₁₋₄ alkyl group;

each X is independently a hydrolysable or hydroxyl group;

R⁵ is a C₁₋₈ alkyl or phenyl group;

v and w are independently 0 or 1, x is 0 or 1 or 2; y is 1 or 2; and z is 2, 3, or 4.

The polyfluoropolyether moiety R^{f} can comprise perfluorinated repeating units selected from the group consisting of —(CₙF₂ₙO)—, —(CF(Z)O)—, —(CF(Z)CₙF₂ₙO)—, —(CₙF₂ₙCF(Z)O)—, —(CF₂CF(Z)O)—, and combinations thereof; wherein n is an integer from 1 to 6 and Z is a perfluoroalkyl group, an oxygen-containing perfluoroalkyl group, a perfluoroalkoxy group, or an oxygen-substituted perfluoroalkoxy group, each of which can be linear, branched, or cyclic, and have 1 to 5 carbon atoms and up to 4 oxygen atoms when oxygen-containing or oxygen-substituted and wherein for repeating units including Z the number of carbon atoms in sequence is at most 6. In particular, n can be an integer from 1 to 4, more particularly from 1 to 3. For repeating units including Z the number of carbon atoms in sequence may be at most four, more particularly at most 3. Usually, n is 1 or 2 and Z is an —CF₃ group, more wherein z is 2, and R^{f} is selected from the group consisting of —CF₂O(CF₂O)ₘ(C₂F₄O)ₚCF₂—, —CF(CF₃)O(CF(CF₃)CF₂O)ₚCF(CF₃)—, —CF₂O(C₂F₄O)ₚCF₂—, —(CF₂)₃O(C₄F₈O)ₚ(CF₂)₃—, —CF(CF₃)—(OCF₂CF(CF₃))ₚO—CₜF₂ₜ—O(CF(CF₃)CF₂O)ₚCF(CF₃)—, wherein t is 2, 3 or 4 and wherein m is 1 to 50, and p is 3 to 40.

A cross-linking agent can be included. Typical cross-linking agents include tetramethoxysilane; tetraethoxysilane; tetrapropoxysilane; tetrabutoxysilane; methyl triethoxysilane;

dimethyldiethoxysilane; octadecyltriethoxysilane; 3-glycidoxy-propyltrimethoxysilane; 3-glycidoxy-propyltriethoxysilane; 3-aminopropyl-trimethoxysilane; 3-aminopropyl-triethoxysilane; bis (3-trimethoxysilylpropyl) amine; 3-aminopropyl tri(methoxyethoxyethoxy) silane; N (2-aminoethyl)3-aminopropyltrimethoxysilane; bis (3-trimethoxysilylpropyl) ethylenediamine; 3-mercaptopropyltrimethoxysilane; 3-mercaptopropyltriethoxysilane; 3-trimethoxysilyl-propylmethacrylate; 3-triethoxysilypropylmethacrylate; bis (trimethoxysilyl) itaconate; allyltriethoxysilane; allyltrimethoxysilane; 3-(N-allylamino)propyltrimethoxysilane; vinyltrimethoxysilane; vinyltriethoxysilane; and mixtures thereof.

The component to be coated can be pre-treated before coating, typically by cleaning. Cleaning can be by way of a solvent, typically a hydrofluoroether, e.g. HFE72DE, or an azeotropic mixture of about 70% w/w trans-dichloroethylene; 30% w/w of a mixture of methyl and ethyl nonafluorobutyl and nonafluoroisobutyl ethers.

The above-described first acceptable coating is particularly useful for coating valves components, including one or more of valve stems, bottle emptiers, springs, and tanks, as well as canisters, such as metered dose inhalers, as described herein. This coating system can be used with any type of inhaler and any formulation described herein.

A second type of coating that can be used comprises a polyphenylsulphone. The polyphenylsulphone typically has the following chemical structure

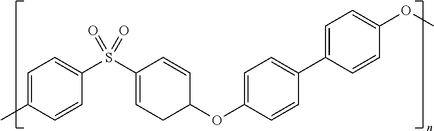

In this structure, n is the number of repeat units, which is typically sufficient to provide a weight average molecular weight from 10,000 to 80,000 daltons, for example, from 10,000 to 30,000 daltons.

Other polymers, such as polyethersulphones, fluoropolymers such as PTFE, FEP, or PFA, can also be included. However, such other polymers are optional, and it is often advantageous to exclude them.

Polyphenylsulphones can be difficult to apply by a solvent casting process. Thus, a special solvent system that is viable for use in a manufacturing setting can be employed for coating the polyphenylsulphones. On such solvent system employs a (1) first solvent that has a Hildebrand Solubility Parameter of at least 20.5 MPa^{0.5} and at most 25 MPa^{0.5}, such as from 21 MPa^{0.5} to 23.5 MPa^{0.5}; and (2) at least 20% by volume, often greater than 70% or greater than 80% by volume, of at least one 5-membered aliphatic, cyclic, or heterocyclic ketone based on the total volume of the solvent system. Optionally, a third component, namely a linear aliphatic ketone, can be included in amounts less than 5% by volume of the total volume of the solvent system.

Any first solvent that has a Hildebrand Solubility Parameter of at least 20.5 MPa^{0.5} and at most 25 MPa^{0.5} can be used, so long as the other components of the solvent system are as stated above. Some such first solvents are also -membered aliphatic, cyclic, or heterocyclic ketones, in which case the first solvent and the -membered aliphatic, cyclic, or heterocyclic ketone can be the same material. Other such solvents include acetonitrile.

The 5-membered aliphatic, cyclic, or heterocyclic ketone is typically a gamma lactone, such as gamma-butyrolactone, or a gamma lactam, such as a pyrolidone like 2-pyrrolidone, or an alkyl substituted 2-pyrrolidone like N-alkyl-2-pyrrolidones such as N-methyl-2-pyrrolidine (sometimes known by the acronym NMP). Other examples of 5-membered aliphatic, cyclic, or heterocyclic ketone that can be used include 2-methyl cyclopentanone, 2-ethyl cyclopentanone, and 2-[1-(5-methyl-2-furyl)butyl]cyclopentanone. Cyclopentanone is the most commonly used material.

The optional linear aliphatic ketone can be any linear aliphatic ketone, and is typically acetone, although methyl ethyl ketone is also frequently employed.

The above-described second acceptable coating can be used on any type of inhaler, but is particularly useful for components of metered dose inhalers.

A third acceptable coating can be used to lower the surface energy of any component of an inhaler, such as a metered dose inhaler, but is particularly useful for valve stems, particularly those made of acetal polymer, as well as for stainless steel or aluminum components, particularly those used in canisters.

Such a coating can be formed on a component of an inhaler by the following process:
a) forming a non-metal coating on at least a portion of a surface of the medicinal inhalation device or a component of a medicinal inhalation device, respectively, said coating having at least one functional group;
b) applying to at least a portion of a surface of the non-metal coating a composition comprising an at least partially fluorinated compound comprising at least one functional group; and
c) allowing at least one functional group of the at least partially fluorinated compound to react with at least one functional group of the non-metal coating to form a covalent bond.

The at least one functional group of the non-metal coating is typically a hydroxyl group or silanol group. In most cases, the non-metal coating has a plurality of functional groups, particularly silanol groups, and can be formed, for example by plasma coating an organosilicone with silanol groups on the inhaler or one or more inhaler components. Typical organosilicon compounds include trimethylsilane, triethylsilane, trimethoxysilane, triethoxysilane, tetramethylsilane, tetraethylsilane, tetramethoxysilane, tetraethoxysilane, hexamethylcyclotrisiloxane, tetramethylcyclotetrasiloxane, tetraethylcyclotetrasiloxane, octamethylcyclotetrasiloxane, hexamethyldisiloxane, bistrimethylsilylmethane, and mixtures thereof. Most commonly, one or more of trimethylsilane, triethylsilane, tetramethylsilane, tetraethylsilane, bistrimethylsilylmethane are employed, with tetramethylsilane being most common. In addition to the organosilicon, the plasma can contain one or more of oxygen, a silicon hydride, particularly silicon tetrahydride, disilane, or a mixture thereof, or both. After deposition, the non-metal coating can be a diamond like glass or carbon like glass containing, on a hydrogen free basis, at 20 atomic percent or more of carbon and 30 atomic percent of more of silicon and oxygen combined.

The non-metal coating is often exposed to an oxygen plasma or corona treatment before applying the partially fluorinated compound. Most typically, an oxygen plasma treatment under ion bombardment conditions is employed.

The at least partially fluorinated compound often contains one or more hydrolysable groups, such as oxyalkly silanes, typically ethyoxy or methoxy silanes. A polyfluoropolyether segment, which in particular cases is a perfluorinated polyfluoroether, is typically used. Poly(perfluoroethylene) glycol is most common. Thus, the at least partially fluorinated compound can include a polyfluropolyether linked to one or more functional silanes by way of, for example, a carbon-silicon, nitrogen-silicon, or sulfer-silicon.

Examples of at least partially fluorinated compounds that can be used include those having the following formula:

R_{f}[Q—[C(R)₂—Si(Y)₃₋ₓ(R^{1a})ₓ]_{y}]_{z}

wherein:
R_{f} is a monovalent or multivalent polyfluoropolyether segment;
Q is an organic divalent or trivalent linking group;
each R is independently hydrogen or a C₁₋₄ alkyl group;
   each Y is independently a hydrolysable group;
R^{1a} is a C₁₋₈ alkyl or phenyl group;
x is 0 or 1 or 2;
   y is 1 or 2; and
z is 1, 2, 3, or 4.

Typically, R_{f}, comprises perfluorinated repeating units selected from the group consisting of —(CₙF₂ₙO)—, —(CF(Z)O)—, —(CF(Z)CₙF₂ₙO)—, —(CₙF₂ₙCF(Z)O)—, —(CF₂CF(Z)O)—, and combinations thereof; wherein n is an integer from 1 to 6 and Z is a perfluoroalkyl group, an oxygen-containing perfluoroalkyl group, a perfluoroalkoxy group, or an oxygen-substituted perfluoroalkoxy group, each of which can be linear, branched, or cyclic, and have 1 to 5 carbon atoms and up to 4 oxygen atoms when oxygen-containing or oxygen-substituted and wherein for repeating units including Z the number of carbon atoms in sequence is at most 6. Particular examples of this compound are those where z is 1, R_{f} is selected from the group consisting of C₃F₇O(CF(CF₃)CF₂O)ₚCF(CF₃)—, CF₃O(C₂F₄O)ₚCF₂—, —C₃F₇O(CF(CF₃)CF₂O)ₚCF₂CF₂—, C₃F₇O(CF₂CF₂CF₂O)ₚCF₂CF₂—, C₃F₇O(CF₂CF₂CF₂O)ₚCF(CF₃)— and CF₃O(CF₂CF(CF₃)O)ₚ(CF₂O)X—, wherein X is CF₂—, C₂F₄—,

C₃F₆—, C₄F₈— and wherein the average value of p is 3 to 50. Other particular examples include those wherein z is 2, R_{f} is selected from the group consisting of —CF₂O(CF₂O)ₘ(C₂F₄O)ₚCF₂—, —CF(CF₃)O(CF(CF₃)CF₂O)ₚCF(CF₃)—, —CF₂O(C₂F₄O)ₚCF₂—, —(CF₂)₃O(C₄F₈O)ₚ(CF₂)₃—, —CF(CF₃)—(OCF₂CF(CF₃))ₚO—C₅F₂ₜ—O(CF(CF₃)CF₂O)ₚCF(CF₃)—, wherein t is 2, 3 or 4 and wherein m is 1 to 50, and p is 3 to 40. Most commonly R_{f} is one of —CF₂O(CF₂O)ₘ(C₂F₄O)ₚCF₂—, —CF₂O(C₂F₄O)ₚCF₂—, and —CF(CF₃)—(OCF₂CF(CF₃))ₚO—(CₜF₂ₜ)—O(CF(CF₃)CF₂O)ₚCF(CF₃)—, t is 2, 3, or 4, and the average value of m+p or p+p or p is from about 4 to about 24. Q is commonly selected from the group consisting of —C(O)N(R)—(CH₂)ₖ—, —S(O)₂N(R)—(CH₂)ₖ—, —(CH₂)ₖ—, —CH₂O—(CH₂)ₖ—, —C(O)S—(CH₂)ₖ—, —CH₂OC(O)N(R)—(CH₂)ₖ—, and

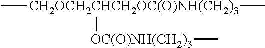

when R is hydrogen or C₁₋₄ alkyl, and k is 2 to about 25. In other common cases, Q is selected from the group consisting of —C(O)N(R)(CH₂)₂—, —OC(O)N(R)(CH₂)₂—, —CH₂O(CH₂)₂—, or —CH₂—OC(O)N(R)—(CH₂)₂—, R is hydrogen or C₁₋₄ alkyl, and y is 1.

Upon applying appropriate at least partially fluorinated compounds to the non-metallic coating, at least one covalent bond can form between the two, thereby completing the coating.

Yet another suitable coating is fluorinated ethylene propylene copolymer, sometimes known as FEP. FEP coatings are particularly useful for coating one or more internal surfaces of a canister, and can be used in association with

### LIST OF EXEMPLARY EMBODIMENTS

The following embodiments are meant to be illustrative, and are not intended to be limiting unless otherwise specified.
1. A composition comprising
   particulate albuterol or a pharmaceutically acceptable salt or solvate thereof;
   particulate ipratropium or a pharmaceutically acceptable salt or solvate thereof at least one of 1,1,1,2,3,3,3-heptafluoropropane and 1,1,1,2-tetrafluoroethane.
2. The composition of embodiment 1, consisting essentially of
   particulate albuterol or a pharmaceutically acceptable salt or solvate thereof;
   ipratropium b or a pharmaceutically acceptable salt or solvate thereof at least one of 1,1,1,2,3,3,3-heptafluoropropane and 1,1,1,2-tetrafluoroethane.
3. The composition of any preceding embodiment, wherein the albuterol or a pharmaceutically acceptable salt or solvate thereof is albuterol sulfate.
4. The composition of any preceding embodiment, wherein the ipratropium or a pharmaceutically acceptable salt or solvate thereof is ipratropium bromide.
5. The composition of embodiment 4 wherein the ipratropium bromide is ipratropium bromide monohydrate.
6. The composition of any preceding embodiment, wherein the canister particle size of the albuterol is no less than 1 micrometer no less than 1.5 micrometers, no less than 2 micrometers, no less than 2.5 micrometers, no less than 3 micrometers, no less than 3.5 micrometers, no less than 4 micrometers, or no less than 4.5 micrometers.
7. The composition of any preceding embodiment, wherein the canister particle size of the albuterol is no greater than 5 micrometers, no greater than 4.5 micrometers, no greater than 4.0 micrometers, no greater than 3.5 micrometers, no greater than 3.0 micrometers, no greater than 2.5 micrometers, no greater than 2.0 micrometers, or no greater than 1.5 micrometers.
8. The composition of any preceding embodiment, wherein the canister size of the ipratropium is no less than 1 micrometer no less than 1.5 micrometers, no less than 2 micrometers, no less than 2.5 micrometers, no less than 3 micrometers, no less than 3.5 micrometers, no less than 4 micrometers, or no less than 4.5 micrometers.
9. The composition of any preceding embodiment, wherein the canister size of the ipratropium is no greater than 5 micrometers, no greater than 4.5 micrometers, no greater than 4.0 micrometers, no greater than 3.5 micrometers, no greater than 3.0 micrometers, no greater than 2.5 micrometers, no greater than 2.0 micrometers, or no greater than 1.5 micrometers.
10. The composition of any preceding embodiment, wherein the concentration of albuterol, on a mg/mL basis, is no less than 4, no less than 4.1, no less than 4.2, no less than 4.3, no less than 4.4, no less than 4.5, no less than 4.6, no less than 4.8, no less than 4.9, no less than 5.0, no less than 5.1, no less than 5.1, no less than 5.2, no less than 5.3, no less than 5.4, no less than 5.5, no less than 5.6, no less than 5.7, no less than 5.8, no less than 5.9, no less than 6.0, no less than 6.1, no less than 6.2, no less than 6.3, no less than 6.4, no less than 6.5, no less than 6.6, no less than 6.7, no less than 6.8, no less than 6.9, no less than 7.0, no less than 7.1, no less than 7.2, no less than 7.3, no less than 7.4, no less than 7.5, no less than 7.6, no less than 7.7, no less than 7.8, no less than 7.9, no less than 8.0, no less than 8.1, no less than 8.2, no less than 8.3, no less than 8.4, no less than 8.5, no less than 8.6, no less than 8.7, no less than 8.8, no less than 8.9, no less than 9.0, no less than 9.1, no less than 9.2, no less than 9.3, no less than 9.4, no less than 9.5, no less than 9.6, no less than 9.7, no less than 9.8, no less than 9.9, no less than 10.0, no less than 10.1, no less than 10.2, no less than 10.3, no less than 10.4, no less than 10.5, no less than 10.6, no less than 10.7, no less than 10.8, no less than 10.9, or no less than 11.
11. The composition of any preceding embodiment, wherein the concentration of albuterol, on a mg/mL basis, is no greater than 11, no greater than 10.9, no greater than 10.8, no greater than 10.7, no greater than 10.6, no greater than 10.5, no greater than 10.4, no greater than 10.3, no greater than 10.2, no greater than 10.1, no greater than 10.0, no greater than 9.9, no greater than 9.8, no greater than 9.7, no greater than 9.6, no greater than 9.5, no greater than 9.4, no greater than 9.3, no greater than 9.2, no greater than 9.1, no greater than 9.0, no greater than 8.9, no greater than 8.8, no greater than 8.7, no greater than 8.6, no greater than 8.5, no greater than 8.4, no greater than 8.3, no greater than 8.2, no greater than 8.1, no greater than 8.0, no greater than 7.9, no greater than 7.8, no greater than 7.7, no greater than 7.6, no greater than 7.5, no greater than 7.4, no greater than 7.3, no greater than 7.2, no greater than 7.1, no greater than 7.0, no greater than 6.9, no greater than 6.8, no greater than 6.7, no greater than 6.6, no greater than 6.5, no greater than 6.4, no greater than 6.3 , no greater than 6.2, no greater than 6.1, no greater than 6.0, no greater than 5.9, no greater than 5.8, no greater than 5.7, no greater than 5.6, no greater than 5.5, no greater than 5.4, no greater than 5.3, no greater than 5.2, no greater than 5.1, no greater than 5.0, no greater than 4.9, no greater than 4.8, no greater than 4.7, no greater than 4.6, no greater than 4.5, no greater than 4.4, no greater than 4.3, no greater than 4.2, or no greater than 4.1.
13. The composition of any preceding embodiment, wherein the concentration of albuterol is from 4 mg/mL to 11 mg/mL.
14. The composition of any preceding embodiment, wherein the concentration of albuterol is from 4.19 mg/mL to 10.56 mg/mL.
15. The composition of any preceding embodiment, wherein the concentration of ipratropium is, on a mg/mL basis, no less than 0.5, no less than 0.6, no less than 0.7, no less than 0.8, no less than 0.9, no less than 1.0, no less than 1.1, no less than 1.2, no less than 1.3, no less than 1.4, no less than 1.5, no less than 1.6, no less than 1.7, no less than 1.8, no less than 1.9, or no less than 2.0.
16. The composition of any preceding embodiment, wherein the concentration of ipratropium is, on a mg/mL basis, no greater than 2.0, no greater than 1.9, no greater than 1.8, no greater than 1.7, no greater than 1.6, no greater than 1.5, no greater than 1.4, no greater than 1.3, no greater than 1.2, no greater than 1.1, no greater than 1.0, no greater than 0.9, no greater than 0.8, no greater than 0.7, no greater than 0.6, or no greater than 0.5.
17. The composition of any preceding embodiment wherein the concentration of ipratropium is from 0.5 mg/mL to 2 mg/mL.
18. The composition of any preceding embodiment wherein the concentration of ipratropium is as from 0.69 mg/mL to 1.76 mg/mL.
19. The composition of any preceding embodiment further comprising one or more surfactants.
20. The composition of any preceding embodiment wherein the one or more surfactants include at least one of oleic acid, sorbitan monooleate, sorbitan trioleate, soya lecithin, polyethylene glycol, and polyvinylpyrrolidone.
21. The composition of embodiment 20, wherein the one or more surfactants include at least one of oleic acid and polyvinylpyrrolidone.
22. The composition of embodiment 21, wherein polyvinylpyrrolidone has a weight average molecular weight from 10 to 100 kilodaltons, optionally from 10 to 50, optionally from 10 to 40 kilodaltons, optionally from 10 to 30 kilodaltons, or optionally 10 to 20 kilodaltons.
23. The composition of any of embodiments 20-22 wherein the surfactant is present, on a weight percent basis, in an amount no less than 0.0001, no less than 0.01, no less than 0.02, no less than 0.03, no less than 0.04, no less than 0.05, no less than 0.06, no less than 0.07, no less than 0.08, no less than 0.09, no less than 0.10, no less than 0.11, no less than 0.12, no less than 0.13, no less than 0.14, no less than 0.15, no less than 0.16, no less than 0.17, no less than 0.18, no less than 0.19, no less than 0.2, no less than 0.21, no less than 0.22, no less than 0.23, no less than 0.24, no less than 0.25, no less than 0.26, no less than 0.27, no less than 0.28, no less than 0.29, no less than 0.3, no less than 0.4, no less than 0.5, no less than 0.6, no less than 0.7, no less than 0.8, no less than 0.9, or no less than 1.
24. The composition of any of embodiments 20-23 wherein the surfactant is present, on a weight percent basis, in an amount no greater than 1, no greater than 0.9, no greater than 0.8, no greater than 0.7, no greater than 0.6, no greater than 0.5, no greater than 0.4, no greater than 0.3, no greater than 0.29, no greater than 0.28, no greater than 0.27, no greater than 0.26, no greater than 0.25, no greater than 0.24, no greater than 0.23, no greater than 0.22, no greater than 0.21, no greater than 0.20, no greater than 0.19, no greater than 0.18, no greater than 0.17, no greater than 0.16, no greater than 0.15, no greater than 0.14, no greater than 0.13, no greater than 0.12, no greater than 0.11, no greater than 0.10, no greater than 0.09, no greater than 0.08, no greater than 0.07, no greater than 0.06, no greater than 0.05, no greater than 0.04, no greater than 0.03, no greater than 0.02, or no greater than 0.01.
24. The composition of any of embodiments 1-23, wherein the surfactant is present from from 0.0001 wt. % to 1 wt. %, optionally 0.001 wt. % to 0.1 wt. %, optionally 0.1 wt. %.
25. The composition of any of the preceding embodiments, further comprising ethanol.
26. The composition of embodiment 25 wherein the weight percent of ethanol is no greater than 5, no greater than 4.9, no greater than 4.8, no greater than 4.7, no greater than 4.6, no greater than 4.5, no greater than 4.4, no greater than 4.3, no greater than 4.2, no greater than 4.1, no greater than 4.0, no greater than 3.9, no greater than 3.8, no greater than 3.7, no greater than 3.6, no greater than 3.5, no greater than 3.4, no greater than 3.3, no greater than 3.2, no greater than 3.1, no greater than 3.0, no greater than 2.9, no greater than 2.8, no greater than 2.7, no greater than 2 6, no greater than 2.5, no greater than 2.4, no greater than 2.3, no greater than 2.2, no greater than 2.1, no greater than 2.0, no greater than 1.9, no greater than 1.8, no greater than 1.7, no greater than 1.6, no greater than 1.5, no greater than 1.4, no greater than 1.3, no greater than 1.2, no greater than 1.1, no greater than 1.0, no greater than 0.9, no greater than 0.8, no greater than 0.7, no greater than 0.6, or no greater than 0.5.
27. The composition of any of embodiments 25-26, wherein the weight percent of ethanol is no less than 0.5, no less than 0.6, no less than 0.7, no less than 0.8, no less than 0.9, no less than 1.0, no less than 1.1, no less than 1.2, no less than 1.3, no less than 1.4, no less than 1.5, no less than 1.6, no less than 1.7, no less than 1.8, no less than 1.9, no less than 2.0, no less than 2.1, no less than 2.2, no less than 2.3, no less than 2.4, no less than 2.5, no less than 2.6, no less than 2.7, no less than 2.8, no less than 2.9, no less than 3.0, no less than 3.1, no less than 3.2, no less than 3.3, no less than 3 .4, no less than 3.5, no less than 3.6, no less than 3.7, no less than 3.8, no less than 3.9, no less than 4 .0, no less than 4.1, no less than 4.2, no less than 4.3, no less than 4.4, no less than 4.5, no less than 4.6, no less than 4.7, no less than 4.8, no less than 4.9, or no less than 5.0.
28. The composition of any of embodiments 25-27 wherein the weight percent of ethanol is from 0.1 wt. % to 5 wt. %, optionally from 0.5 wt. % to 4 wt. %.
29. The composition of embodiment 28, wherein the weight percent of ethanol is 1 wt. % is employed.
30. The composition of any preceding embodiment wherein the albuterol concentration is 4.19 mg/mL.
31. The composition of any of embodiments 1-29 wherein the albuterol concentration is 5.28 mg/mL.
32. The composition of any of embodiments 1-29 wherein the albuterol concentration is 10.56 mg/mL.
33. The composition of any of embodiments 30-32 wherein the albuterol is albuterol sulfate.
34. The composition of any preceding embodiment wherein the concentration of ipratropium is 0.69 mg/mL.
35. The composition of any of embodiments 1-33 wherein the concentration of ipratropium is 0.88 mg/mL.
36. The composition of any of embodiments 1-33 wherein the concentration of ipratropium is 1.76 mg/mL.
37. The composition of any of embodiments 34-36 wherein the ipratropium is ipratropium bromide.
38. The composition of embodiment 37 wherein the ipratropium bromide is ipratropium bromide monohydrate.
39. The composition of any preceding embodiment wherein the composition is stable after six months of storage inside an aerosol canister at a temperature of 40° C. and a relative humidity of 75%.
40. The composition of any preceding embodiment, wherein either
   a) the change in fine particle size after storage for six months in an aerosol canister fitted with an actuator is no greater than 15%, no greater than 14%, no greater than 13%, no greater than 12%, no greater than 11%, no greater than 10%, no greater than 9%, no greater than 8%, no greater than 7%, no greater than 6%, no greater than 5%, no greater than 4%, no greater than 3%, no greater than 2%, or no greater than 1%; or
   b) the change in fine particle mass after storage for six months in an aerosol canister fitted with an actuator is no greater than 15%, no greater than 14%, no greater than 13%, no greater than 12%, no greater than 11%, no greater than 10%, no greater than 9%, no greater than 8%, no greater than 7%, no greater than 6%, no greater than 5%, no greater than 4%, no greater than 3%, no greater than 2%, or no greater than 1%; or
   c) both a) and b).
41. The composition of embodiment 40 wherein the change in fine particle size is no greater than 5%, or wherein the change in fine particle mass is no greater than 5%, or wherein the change in fine particle size and fine particle mass are both no greater than 5%.
42. An aerosol canister containing a formulation of any preceding embodiment.
43. The aerosol canister of embodiment 42 comprising at least one surface having a primer composition comprising a silane having two or more reactive silane groups separated by an organic linker group disposed thereon, wherein the primer composition has a coating composition comprising an at least partially fluorinated compound disposed thereon.
44. The aerosol canister of embodiment 43, wherein the at least partially fluorinated compound is an at least partially fluorinated polyethersilane.
45. An aerosol canister of embodiment 42 comprising at least one surface having a coating comprising polyphenylsulphone.
46. An aerosol canister of embodiment 42 comprising at least one surface having a coating comprising a diamond like glass or carbon like glass.
47. An inhaler comprising the formulation of any of embodiments 1-39 or the aerosol canister of embodiment 40.
48. The inhaler of embodiment 47 that is a metered dose inhaler.
49. The inhaler of any of embodiments 47-48 comprising a valve stem.
50. The inhaler of any of embodiments 47-49 comprising a dose counter.

### EXAMPLES

HFA-134a (1,1,1,2-tetrafluoroethane) and HFA-227 (1,1,1,2,3,3,3-heptafluoropropane) were obtained from the DuPont Corporation (Wilmington, Del.) or the Solvay Corporation (Brussels, Belgium). Ipratropium bromide monohydrate was obtained from Vamsi Labs Ltd. (Solapur, India) or Sifavitor-Infa Group (Milan, Italy). Albuterol sulphate was obtained Vamsi Labs Ltd. (Solapur, India) or Teva Pharmaceutical Products Ltd. (North Wales, Pa.). Ethanol was obtained from Hayman Specialty Products (Witham, England), Sigma-Aldrich Corporation (St. Louis, Mo.), or Pharmco-AAPER (Brookfield, Conn.). Span-85 (sorbitan trioleate) and PVP-10 (polyvinylpyrrolidone of average molecular weight 10,000) were obtained from Sigma-Aldrich Corporation (St. Louis, Mo.). Oleic acid was obtained from Merck-Millipore (Darmstadt, Germany).

### Example 1

Metered dose inhalers (MDIs) were prepared using 15 mL deep drawn aluminum canisters (3M Corporation, Clitheroe, UK), 50 microliter SPRAYMISER type valves fitted with EPDM (ethylene-propylene diene terpolymer elastomer) diaphragm seals (3M Corporation), and 0.6 mm orifice diameter actuators (part No. HP-22817-1MA, RPC-Formatec GmbH, Mellrichstadt, Germany). Albuterol sulfate and ipratropium bromide monohydrate were each micronized to provide a mass median diameter (MMD) range of about 1-5 microns. The canisters were cold filled with the suspension formulation of Table 1. The bulk formulation for cold filling individual canisters was prepared by combining albuterol sulfate and ipratropium bromide monohydrate with a portion of the HFA-227 propellant (about half of the total propellant) in a vessel chilled to less than −50° C. The suspension was high shear mixed for 5-10 minutes using a Silverson mixer (Silverson, East Longmeadow, Mass.). The remaining propellant was then added to the chilled vessel and high shear mixing was continued for an additional 10 minutes.

### Example 2

Metered dose inhalers were prepared according to the description of Example 1 with the exception that the internal surface of each canister was coated with FEP (fluorinated ethylene propylene copolymer).

TABLE 1
Suspension Formulation of Examples 1 and 2

| Formulation Ingredient | Amount (in Percent by Weight) |
|---|---|
| Albuterol sulfate | 0.335 |
| Ipratropium Bromide Monohydrate | 0.060 |
| HFA-227 | 99.605 |

### Next Generation Impactor (NGI) Studies

The aerodynamic particle size distribution emitted from each MDI was evaluated using a Next Generation Impactor Instrument (MSP Corporation, Shoreview, Minn.). For each test, an MDI was attached to the throat component (USP Inlet) of the NGI instrument and actuated 10 times into the instrument. Immediately prior to attachment, the MDI was primed by actuating 3 times. The flow rate through the instrument during testing was regulated at 30 L/minute. The test sample (albuterol sulfate and ipratropium bromide) deposited on the valve stem, actuator, throat assembly (USP inlet), individual collection cups 1-7, micro-orifice collector (MOC), and final filter component was collected by rinsing each individual component with a known volume of collection solvent (30/70 (v/v) acidified water (pH 2.6, H₃PO₄)/acetonitrile). The recovered samples were then analyzed for sample content using an HPLC assay with a reference standard curve. An Agilent 1200 HPLC instrument with a UV detector (220 nm) and a Zorbax SB-C18, micron, 4.6-150 mm column (40° C. column temp) was used (Agilent Technologies, Santa Clara, Calif.). A 2-component gradient mobile phase was used with 0.2% aqueous HClO₄ as the first component and acetonitrile as the second component (gradient 5% acetonitrile to 90% acetonitrile). The injection volume was 20 microliters and the flow rate was 1 mL/min.
The MDIs were stored in an inverted orientation (i.e. valve positioned down) in a 4° C./40° C. cycling chamber (cycling rate 6 hours) for a period of either 5, 6 or 9 weeks. MDIs were tested on the day prepared (Day 0) and after either 5, 6 or 9 weeks of storage. In Table 2, the total Ex-valve content, fine particle mass (FPM), and fine particle fraction (FPF) data for albuterol sulfate is presented. In Table 3, the corresponding data for ipratropium bromide is presented. At each time point three individual MDIs were tested and the result is presented as the mean value with standard deviation [SD].
Total Ex-valve content was determined as the sum of the sample content from all of the twelve analyzed components (valve stem through filter) (reported as micrograms/actuation).
FPM (Fine Particle Mass) was determined as the sum of the sample content determined for Cups 3-7, the MOC, and the filter (reported as micrograms/actuation).
FPF (Fine Particle Fraction)=[FPM/(sum of sample content for throat assembly, cups 1-7, MOC, and the filter)]×100.

TABLE 2
NGI Particle Data for Albuterol sulfate

| | | Total Ex-Valve | | | | |
|---|---|---|---|---|---|---|
| | | Content | | FPM | | FPF |
| | | mcg/actuation | | mcg/actuation | | % |
| MDI | Test Point | [SD] | | [SD] | | [SD] |
| Example 1 | Initial (Day 0) | 189.1 | [0.6] | 109.4 | [1.0] | 65.6 [1.3] |
| Example 1 | 5 weeks | 175.9 | [7.7] | 98.1 | [4.4] | 63.8 [0.9] |
| Example 2 | 5 weeks | 175.8 | [20] | 100.9 | [11.3] | 64.4 [0.7] |

TABLE 3
NGI Particle Data for Ipratropium Bromide

| | | Total Ex-Valve | | |
|---|---|---|---|---|
| | | Content | FPM | FPF |
| | | mcg/actuation | mcg/actuation | % |
| MDI | Test Point | [SD] | [SD] | [SD] |
| Example 1 | Initial (Day 0) | 31.4 [4.6] | 15.4 [2.8] | 56.0 [1.1] |
| Example 1 | 5 weeks | 29.2 [0.9] | 13.9 [0.6] | 53.8 [0.4] |
| Example 2 | 5 weeks | 28.8 [3.6] | 14.4 [1.8] | 55.2 [0.4] |

### Example 3

Metered dose inhalers (MDIs) were prepared according to the general procedure described in Example 1 using the formulation of Table 4 with the exception that 50 microliter 3M Retention valves with an EPDM elastomer seal (3M Corporation, Clitheroe, UK) and actuators with a 0.5 mm orifice were used. The MDIs were stored in an inverted orientation in a 4° C./40° C. cycling chamber (cycling rate 6 hours) for a period of 9 weeks. MDIs were tested for ex-actuator particle size distribution on the day prepared (Day 0) and after 9 weeks of storage using the NGI study procedure described above. In Table 5, the amount of albuterol sulfate (micrograms/actuation) recovered from the valve stem, actuator, and each stage of the NGI instrument (throat assembly, cups 1-7, MOC, filter) is presented. In Table 6, the corresponding particle size distribution data for ipratropium bromide is presented.

TABLE 4
Suspension Formulation of Example 3

| Formulation Ingredient | Amount (in Percent by Weight) |
|---|---|
| Albuterol sulfate | 0.173 |
| Ipratropium Bromide Monohydrate | 0.030 |
| HFA-227 | 99.797 |

TABLE 5
NGI Test Results for Albuterol sulfate (MDI of Example 3)

| | Initial (Day 0) | 9 weeks |
|---|---|---|
| Test Component | (mcg/actuation) | (mcg/actuation) |
| Valve Stem | 12.20 | 14.00 |
| Actuator | 13.60 | 14.50 |
| Throat (USP Inlet) | 28.30 | 28.30 |
| Cup 1 | 0.42 | 0.63 |
| Cup 2 | 1.29 | 1.51 |
| Cup 3 | 5.22 | 5.50 |
| Cup 4 | 23.10 | 21.30 |
| Cup 5 | 21.40 | 18.80 |
| Cup 6 | 4.06 | 3.42 |
| Cup 7 | 1.01 | 1.17 |
| MOC | 0.74 | 0.63 |
| Filter | 2.18 | 3.14 |

TABLE 6
NGI Test Results for Ipratropium Bromide (MDI of Example 3)

| | Initial (Day 0) | 9 weeks |
|---|---|---|
| Test Component | (mcg/actuation) | (mcg/actuation) |
| Valve Stem | 1.83 | 2.24 |
| Actuator | 2.74 | 2.77 |
| Throat (USP Inlet) | 5.92 | 6.65 |
| Cup 1 | 0.14 | 0.17 |
| Cup 2 | 0.61 | 0.68 |
| Cup 3 | 1.70 | 1.72 |
| Cup 4 | 3.71 | 3.39 |
| Cup 5 | 1.94 | 1.79 |
| Cup 6 | 0.30 | 0.30 |
| Cup 7 | 0.09 | 0.13 |
| MOC | 0.08 | 0.09 |
| Filter | 0.09 | 0.15 |

### Example 4

Metered dose inhalers (MDIs) were prepared according to the general procedure described in Example 3 using the formulation of Table 7. HFA-134a was used as the propellant in place of HFA-227. The MDIs were stored in an inverted orientation in a 4° C./40° C. cycling chamber (cycling rate 6 hours) for a period of 6 weeks. MDIs were tested for particle size distribution on the day prepared (Day 0) and after 6 weeks of storage using the NGI study procedure described above. In Table 8, the amount of albuterol sulfate (micrograms/actuation) recovered from the valve stem, actuator, and each stage of the NGI instrument (throat assembly, cups 1-7, MOC, filter) is presented. In Table 9, the corresponding particle size distribution data for ipratropium bromide is presented.

TABLE 7
Suspension Formulation of Example 4

| Formulation Ingredient | Amount (in Percent by Weight) |
|---|---|
| Albuterol sulfate | 0.200 |
| Ipratropium Bromide Monohydrate | 0.035 |
| HFA-134a | 99.765 |

TABLE 8
NGI Test Results for Albuterol sulfate (MDI of Example 4)

| | Initial (Day 0) | 6 weeks |
|---|---|---|
| Test Component | (mcg/actuation) | (mcg/actuation) |
| Valve Stem | 19.94 | 19.24 |
| Actuator | 17.01 | 15.87 |
| Throat (USP Inlet) | 33.19 | 28.09 |
| Cup 1 | 0.47 | 0.57 |
| Cup 2 | 0.79 | 0.81 |
| Cup 3 | 3.67 | 3.60 |
| Cup 4 | 21.75 | 20.03 |
| Cup 5 | 22.98 | 21.84 |
| Cup 6 | 4.81 | 4.79 |
| Cup 7 | 1.25 | 1.41 |
| MOC | 0.94 | 0.93 |
| Filter | 2.25 | 3.62 |

TABLE 9
NGI Test Results for Ipratropium Bromide (MDI of Example 4)

| | Initial (Day 0) | 6 weeks |
|---|---|---|
| Test Component | (mcg/actuation) | (mcg/actuation) |
| Valve Stem | 3.19 | 3.32 |
| Actuator | 3.42 | 3.06 |
| Throat (USP Inlet) | 7.90 | 6.58 |
| Cup 1 | 0.10 | 0.13 |
| Cup 2 | 0.25 | 0.24 |
| Cup 3 | 1.20 | 1.14 |
| Cup 4 | 4.32 | 4.00 |
| Cup 5 | 2.87 | 2.70 |
| Cup 6 | 0.51 | 0.53 |
| Cup 7 | 0.17 | 0.22 |
| MOC | 0.16 | 0.19 |
| Filter | 0.22 | 0.27 |

### Example 5

Metered dose inhalers (MDIs) were prepared according to the general procedure described in Example 3 using the formulation of Table 10. The bulk formulation for cold filling individual canisters was prepared by combining albuterol sulfate and ipratropium bromide monohydrate with a portion of the HFA-227 propellant (about half of the total propellant) in a vessel chilled to less than −50° C. The suspension was high shear mixed for 5-10 minutes using a Silverson mixer. The remaining propellant, PVP-10 (polyvinylpyrrolidone, weight average molecular weight 10 kilodaltons), and ethanol were then added to the chilled vessel and mixing was continued for an additional 10 minutes. The MDIs were stored in an inverted orientation in a 4° C./40° C. cycling chamber (cycling rate 6 hours) for a period of 6 weeks. MDIs were tested for particle size distribution on the day prepared (Day 0) and after 6 weeks of storage using the NGI study procedure described above. In Table 11, the amount of albuterol sulfate (micrograms/actuation) recovered from the valve stem, actuator, and each stage of the NGI instrument (throat assembly, cups 1-7, MOC, filter) is presented. In Table 12, the corresponding particle size distribution data for ipratropium bromide is presented.

TABLE 10
Suspension Formulation of Example 5

| Formulation Ingredient | Amount (in Percent by Weight) |
|---|---|
| Albuterol sulfate | 0.173 |
| Ipratropium Bromide Monohydrate | 0.030 |
| PVP-10 | 0.010 |
| Ethanol | 1.000 |
| HFA-227 | 98.787 |

TABLE 11
NGI Test Results for Albuterol sulfate (MDI of Example 5)

| | Initial (Day 0) | 6 weeks |
|---|---|---|
| Test Component | (mcg/actuation) | (mcg/actuation) |
| Valve Stem | 13.41 | 14.26 |
| Actuator | 14.41 | 15.16 |
| Throat (USP Inlet) | 23.16 | 23.08 |
| Cup 1 | 0.62 | 0.62 |
| Cup 2 | 1.07 | 1.27 |
| Cup 3 | 5.00 | 5.67 |
| Cup 4 | 24.66 | 24.09 |
| Cup 5 | 23.90 | 21.11 |
| Cup 6 | 4.97 | 4.27 |
| Cup 7 | 1.66 | 1.60 |
| MOC | 0.99 | 1.08 |
| Filter | 3.99 | 3.15 |

TABLE 12
NGI Test Results for Ipratropium Bromide (MDI of Example 5)

| | Initial (Day 0) | 6 weeks |
|---|---|---|
| Test Component | (mcg/actuation) | (mcg/actuation) |
| Valve Stem | 2.37 | 2.46 |
| Actuator | 2.60 | 2.76 |
| Throat (USP Inlet) | 4.73 | 5.00 |
| Cup 1 | 0.13 | 0.16 |
| Cup 2 | 0.31 | 0.41 |
| Cup 3 | 1.44 | 1.66 |
| Cup 4 | 4.88 | 4.25 |
| Cup 5 | 3.00 | 2.24 |
| Cup 6 | 0.50 | 0.38 |
| Cup 7 | 0.23 | 0.19 |
| MOC | 0.20 | 0.19 |
| Filter | 0.34 | 0.24 |

### Example 6

Metered dose inhalers (MDIs) were prepared according to the general procedure described in Example 5 using the formulation of Table 13. The bulk formulation for cold filling individual canisters was prepared by combining albuterol sulfate and ipratropium bromide monohydrate with a portion of the HFA-227 propellant (about half of the total propellant) in a vessel chilled to less than −50° C. The suspension was high shear mixed for 5-10 minutes using a Silverson mixer (Silverson, East Longmeadow, Mass.). The remaining propellant, Span-85 (sorbitan trioleate), and ethanol were then added to the chilled vessel and mixing was continued for an additional 10 minutes. The MDIs were stored in an inverted orientation in a 4° C./40° C. cycling chamber (cycling rate 6 hours) for a period of 6 weeks. MDIs were tested for particle size distribution on the day prepared (Day 0) and after 6 weeks of storage using the NGI study procedure described above. In Table 14, the total Ex-valve content (micrograms/actuation) and FPM (micrograms/actuation) are presented for albuterol sulfate. In Table 15, the corresponding data for ipratropium bromide is presented.

TABLE 13
Suspension Formulation of Example 6

| Formulation Ingredient | Amount (in Percent by Weight) |
|---|---|
| Albuterol sulfate | 0.173 |
| Ipratropium Bromide Monohydrate | 0.030 |
| Span-85 | 0.010 |
| Ethanol | 1.000 |
| HFA-227 | 98.787 |

TABLE 14
NG1 Particle Data for Albuterol sulfate

| | | Total Ex-Valve | |
|---|---|---|---|
| | | Content | FPM |
| MDI | Test Point | (mcg/actuation) | (mcg/actuation) |
| Example 6 | Initial (Day 0) | 61.5 | 40.25 |
| Example 6 | 6 weeks | 54.31 | 37.31 |

TABLE 15
NG1 Particle Data for Ipratropium Bromide

| | | Total Ex-Valve | |
|---|---|---|---|
| | | Content | FPM |
| MDI | Test Point | (mcg/actuation) | (mcg/actuation) |
| Example 6 | Initial (Day 0) | 11.20 | 6.80 |
| Example 6 | 6 weeks | 9.70 | 6.06 |

### Example 7

Metered dose inhalers (MDIs) were prepared using a 15 mL deep drawn aluminum canister (internal surface coated with FEP), a 60 microliter 3M Retention valve with an EPDM elastomer seal, and a 0.4 mm orifice diameter actuator with a 0.8 mm jet length. Albuterol sulfate and ipratropium bromide monohydrate were each micronized to provide a mass median diameter (MMD) range of about 1-5 microns. The canisters were cold filled with a suspension formulation composed of albuterol sulfate (0.312 weight percent), ipratropium bromide monohydrate (0.052 weight percent), and HFA-227 (99.636 weight percent). MDIs were cold filled with the formulation according to the filling procedure described in Example 1.

### Example 8

Metered dose inhalers (MDIs) were prepared using a 15 mL deep drawn aluminum canister (internal surface coated with FEP), a 60 microliter 3M Retention valve with an EPDM elastomer seal, and a 0.4 mm orifice diameter actuator with a 0.8 mm jet length. Albuterol sulfate and ipratropium bromide monohydrate were each micronized to provide a MMD range of about 1-5 microns. The canisters were cold filled with a suspension formulation composed of albuterol sulfate (0.314 weight percent), ipratropium bromide monohydrate (0.052 weight percent), oleic acid (0.010 weight percent), ethanol (1.000 weight percent) and HFA-227 (98.624 weight percent). MDIs were cold filled with the formulation according to the filling procedure described in Example 5 with oleic acid replacing PVP-10 as the surfactant.

### Example 9

Metered dose inhalers (MDIs) can be prepared using a 15 mL deep drawn aluminum canister (internal surface coated with FEP), a 50 microliter 3M Retention valve with an EPDM elastomer seal, and a 0.4 mm orifice diameter actuator with a 0.8 mm jet length. Albuterol sulfate and ipratropium bromide monohydrate can each be micronized to provide a MMD range of about 1-5 microns. The canisters can be cold filled with a suspension formulation composed of albuterol sulfate (0.375 weight percent), ipratropium bromide monohydrate (0.062 weight percent), and HFA-227 (99.563 weight percent). MDIs can be cold filled with the formulation according to the filling procedure described in Example 1.

### Example 10

Metered dose inhalers (MDIs) can be prepared using a 15 mL deep drawn aluminum canister (internal surface coated with FEP), a 50 microliter 3M Retention valve with an EPDM elastomer seal, and a 0.4 mm orifice diameter actuator with a 0.8 mm jet length. Albuterol sulfate and ipratropium bromide monohydrate can each be micronized to provide a MMD range of about 1-5 microns. The canisters can be cold filled with a suspension formulation composed of albuterol sulfate (0.377 weight percent), ipratropium bromide monohydrate (0.063 weight percent), oleic acid (0.010 weight percent), ethanol (1.000 weight percent) and HFA-227 (98.550 weight percent). MDIs can be cold filled with the formulation according to the general filling procedure described in Example 5 with oleic acid replacing PVP-10 as the surfactant.

### Example 11

Metered dose inhalers (MDIs) can be prepared using a 15 mL deep drawn aluminum canister (internal surface coated with FEP), a 63 microliter 3M Retention valve with an EPDM elastomer seal, and a 0.4 mm orifice diameter actuator with a 0.8 mm jet length. Albuterol sulfate and ipratropium bromide monohydrate can each be micronized to provide a MMD range of about 1-5 microns. The canisters can be cold filled with a suspension formulation composed of albuterol sulfate (0.297 weight percent), ipratropium bromide monohydrate (0.050 weight percent), and HFA-227 (99.653 weight percent). MDIs can be cold filled with the formulation according to the filling procedure described in Example 1.

### Example 12

Metered dose inhalers (MDIs) can be prepared using a 15 mL deep drawn aluminum canister (internal surface coated with FEP), a 63 microliter 3M Retention valve with an EPDM elastomer seal, and a 0.4 mm orifice diameter actuator with a 0.8 mm jet length. Albuterol sulfate and ipratropium bromide monohydrate can each be micronized to provide a MMD range of about 1-5 microns. The canisters can be cold filled with a suspension formulation composed of albuterol sulfate (0.299 weight percent), ipratropium bromide monohydrate (0.050 weight percent), oleic acid (0.010 weight percent), ethanol 1.000 weight percent) and HFA-227 (98.641 weight percent). MDIs can be cold filled with the formulation according to the filling procedure described in Example 5 with oleic acid replacing PVP-10 as the surfactant.

### Example 13

Suspension formulations A-M for use in metered dose inhalers can be prepared with the compositions reported in Table 16. The content of each component in a formulation is reported as the weight percent. Albuterol sulfate and ipratropium bromide monohydrate are each micronized to provide a MMD range of about 1-5 microns.

TABLE 16
Suspension Formulations

Weight Percent of the Formulation Component

| | Albuterol | Ipratropium Bromide | |
|---|---|---|---|
| Formulation | sulfate | Monohydrate | HFA-227 |
| A | 0.375 | 0.062 | 99.563 |
| B | 0.187 | 0.031 | 99.782 |
| C | 0.156 | 0.026 | 99.818 |
| D | 0.149 | 0.025 | 99.826 |
| E | 0.312 | 0.060 | 99.628 |
| F | 0.156 | 0.030 | 99.814 |
| G | 0.130 | 0.025 | 99.845 |
| H | 0.124 | 0.024 | 99.852 |
| I | 0.749 | 0.125 | 99.126 |
| J | 0.625 | 0.120 | 99.255 |
| K | 0.312 | 0.060 | 99.628 |
| L | 0.260 | 0.050 | 99.690 |
| M | 0.248 | 0.048 | 99.704 |

### Example 14

Suspension formulations N-Z for use in metered dose inhalers can be prepared with the compositions reported in Table 17. The content of each component in a formulation is reported as the weight percent. Albuterol sulfate and ipratropium bromide monohydrate are each micronized to provide a MMD range of about 1-5 microns.

TABLE 17
Suspension Formulations

| | Weight Percent of the Formulation Component | | | | |
|---|---|---|---|---|---|
| | Albuterol | Ipratropium | | | |
| | sulfate | Bromide | | | |
| Formulation | sulfate | Monohydrate | Oleic Acid | Ethanol | HFA-227 |
| N | 0.377 | 0.063 | 0.010 | 1.000 | 98.550 |
| O | 0.189 | 0.031 | 0.010 | 1.000 | 98.770 |
| P | 0.157 | 0.026 | 0.010 | 1.000 | 98.807 |
| Q | 0.150 | 0.025 | 0.010 | 1.000 | 98.815 |
| R | 0.314 | 0.060 | 0.010 | 1.000 | 98.616 |
| S | 0.157 | 0.030 | 0.010 | 1.000 | 98.803 |
| T | 0.131 | 0.025 | 0.010 | 1.000 | 98.834 |
| U | 0.125 | 0.024 | 0.010 | 1.000 | 98.841 |
| V | 0.755 | 0.126 | 0.010 | 1.000 | 98.109 |
| W | 0.629 | 0.121 | 0.010 | 1.000 | 98.240 |
| X | 0.314 | 0.060 | 0.010 | 1.000 | 98.616 |
| Y | 0.262 | 0.050 | 0.010 | 1.000 | 98.678 |
| Z | 0.250 | 0.048 | 0.010 | 1.000 | 98.692 |

1. A composition comprising
particulate albuterol or a pharmaceutically acceptable salt or solvate thereof;
particulate ipratropium or a pharmaceutically acceptable salt or solvate thereof at least one of 1,1,1,2,3,3,3-heptafluoropropane and 1,1,1,2-tetrafluoroethane.

2. The composition of claim 1, wherein the albuterol or a pharmaceutically acceptable salt or solvate thereof is albuterol sulfate.

3. The composition of claim 1 wherein the ipratropium bromide is ipratropium bromide monohydrate.

4. The composition of claim 1 wherein the concentration of albuterol is from 4 mg/mL to 11 mg/mL.

5. The composition of claim 1 wherein the concentration of ipratropium is from 0.5 mg/mL to 2 mg/mL.

6. The composition of claim 1 further comprising one or more surfactants.

7. The composition of claim 6 wherein the one or more surfactants include at least one of oleic acid, sorbitan monooleate, sorbitan trioleate, soya lecithin, polyethylene glycol, and polyvinylpyrrolidone.

8. The composition of claim 6 wherein the surfactant is present from from 0.0001 wt. % to 1 wt. %, optionally 0.001 wt. % to 0.1 wt. %, optionally 0.1 wt. %.

9. The composition of claim 1, further comprising ethanol.

10. The composition of claim 9 wherein the weight percent of ethanol is from 0.1 wt. % to 5 wt %.

11. The composition of claim 1 wherein the albuterol concentration is 4.19 mg/mL.

12. The composition of claim 1 wherein the concentration of ipratropium is 0.69 mg/mL.

13. An aerosol canister comprising a composition of claim 1.

14. An inhaler comprising the aerosol canister of claim 13.

15. An inhaler of claim 14 that is a metered dose inhaler.
